(19) United States
(12) Patent Application Publication
(10) Pub. No.: US 2014/0322742 A1
(43) Pub. Date: Oct. 30, 2014

(54) METHODS AND A DEVICE FOR THE FORMATION OF THREE-DIMENSIONAL MULTICELLULAR ASSEMBLIES

(71) Applicants: CONSEJO SUPERIOR DE INVESTIGATIONES CIENTIFICAS, Madrid (ES); CYTOO, Grenoble (FR)

(72) Inventors: Muriel Auzan, Echirolles (FR); Michel Bornens, Sceaux (FR); Fernando Martin-Belmonte, Madrid (ES); Alejo Ezequiel Rodriguez Fraticelli, Madrid (ES); Joanne Young, Le Chevalon De Voreppe (FR)

(73) Assignees: CYTOO, Grenoble (FR); CONSEJO SUPERIOR DE INVESTIGATIONES CIENTIFCAS, Madrid (ES)

(21) Appl. No.: 14/234,522

(22) PCT Filed: Jul. 24, 2012
(86) PCT No.: PCT/EP2012/064518

§ 371 Date: May 9, 2014

Related U.S. Application Data
(60) Provisional application No. 61/511,232, filed on Jul. 25, 2011.

(30) Foreign Application Priority Data
Jul. 25, 2011 (EP) 11305965.3

(51) Int. Cl. G01N 33/50

(52) U.S. Cl. CPC G01N 33/5005
USPC 435/29

(57) ABSTRACT

The present invention relates to devices and associated methods for forming three-dimensional multicellular assemblies in vitro. Specifically, the present invention relates to devices comprising at least one three-dimensional multicellular assembly immobilised on a two-dimensional adhesive pattern, wherein said three-dimensional multicellular assembly has an organised structure with a normalised polarity and methods for the formation of three-dimensional multicellular assemblies having an organised structure.

### FIELD OF THE INVENTION

The present invention relates to devices and associated methods for forming three-dimensional multicellular assemblies in vitro. Specifically, the present invention relates to devices and methods for the formation of three-dimensional multicellular assemblies having an organised structure.

### BACKGROUND

Cell physiology and pathophysiology has been traditionally studied using either in vivo animal examination or in vitro cell culture of cells or cell lines. When grown on two-dimensional supports, such as plastic dishes or glass coverslips, cells tend to form a monolayer. More recently, in vitro culture of cells on supports treated with extracellular matrix gels (such as collagen gels or Matrigel™, or a combination of both) has proven the ability of cells to grow into three-dimensional multicellular assemblies, which can develop closed lumens, thus acquiring a more physiologically relevant architecture. These assemblies are more generally called organoids, and the culture systems are usually referred to as organotypic cell culture.

Organoid cultures not only resemble their original tissues in their architecture, but also in their function. Indeed, many pathways and molecules, which are not required in two-dimensional monolayer cell culture, are crucial for organoid growth. As such, the organotypic culture systems are more resilient to cell death in comparison to two-dimensional monolayer cell culture. Thus, many drugs that have been excluded from important screenings and trials because of high renal or hepatic cell toxicity using two-dimensional cell-based assays may not have affected a three-dimensional cellular organization. Conversely, many drugs that do not elicit a cytotoxic response in monolayer cell culture may modify aspects of organ physiology (for example, ion channel transport and paracellular water transport), which can only be studied in a three-dimensional culture system. Furthermore, many pathological conditions, such as epithelial-mesenchymal transition or lumen occlusion, are features that are only physiologically relevant in organoid-cultured cells. Accordingly, organoid culture is useful in a variety of applications, including diagnosis, transplantation and drug development (Herlyn et al. U.S. Pat. No. 7,217,570 B2).

However, there are a number of problems with current organoid cell culture methods. Chiefly, organoid cell culture is performed in an environment where there is no control over organoid formation. Organoid structure, including lumen formation, is random, sporadic and unpredictable, organoids do not resemble each other and rapid analysis of high-content is difficult or not possible. Accordingly, there have been attempts to standardization organoid growth (Kataoka et al. U.S. Patent 2004/0197907; Kataoka et al. U.S. Patent 2010/7691369B2; Sokabe et al. U.S. Patent 2010/0331216A1; Fang et al. U.S. Patent 2009/0298166A1).

For example, document US 2010/0331216A1 discloses a cell culture container comprising an array of square wells delimited by sidewalls, wherein each sidewall has a central opening that allows communication between adjacent wells.

The size of the central openings is defined so as to minimize the contact of the cells of a well with the cells of an adjacent well.

However, in such a container, the cells are formed into multiple layers in each well and thus do not have an organized structure.

Another method for forming three-dimensional multicellular assemblies is disclosed by F. Xu et al. (“A three-dimensional in vitro ovarian cancer coculture model using a high-throughput cell patterning platform”, Biotechnol. J. 2011, 6, 204-212).

This method consists in coating a culture dish with Matrigel™ and in depositing individual cell-encapsulating droplets at specific locations on the Matrigel™.

However, the organization of the cellular assemblies within each droplet is not controlled and the cellular assemblies may organize in different ways from a droplet to another one.

In order to better control the culture of three-dimensional multicellular assemblies, C. M. Williams et al. disclose a micromolded PEG-fibrinogen hydrogel support comprising an array of micro-wells (“Autocrine-Controlled Formation and Function of Tissue-Like Aggregates by Primary Hepatocytes in Micropatterned Hydrogel Arrays”, Tissue Engineering: Part A, Vol. 17, Nos 7 and 8, 2011).

Although the micro-wells were non-adhesive for hepatocytes, primary hepatocytes were grown in each micro-well and aggregated into three-dimensional assemblies, whose cell-secreted matrix was able to adhere to the walls of the micro-wells.

However, as mentioned in this paper, culture of primary hepatocytes on two-dimensional (i.e. non-molded) hydrogels were unsuccessful, since cells did not attach to the support and only formed floating spheroids.

While current three-dimensional culture systems provide organoid formation with an efficient yield, automated imaging and data acquisition remains complicated and as such the use of these organoids for high-throughput experimental studies remains difficult.

Firstly, current assemblies are generally of a thickness of more than 20 μm. High resolution imaging of such assemblies requires confocal or two-photon excitation microscopy with z section stacking, which significantly increases the time and data volume of the image acquisition process. Additionally, the organoids are often embedded in gel.

During cell seeding and three-dimensional growth, the organoids are randomly dispatched in different planes throughout the gel. This necessitates large imaging volumes and high resource-consuming data mining.

Secondly, the method is hampered by a high level of noise. In other words, because organoid formation is random and sporadic, the cells of the formed organoids are not organized but distributed randomly within the structure. Accordingly, organoids do not resemble each other in terms of shape, size, structure and cell positioning. Such variability does not allow for automation of the qualitative and quantitative analysis required for high-throughput experimental studies. Further, measurements suffer from a lack of reproducibility. In addition, because organoid formation is random and sporadic, the structure of the organoid cannot be controlled. Therefore, an acinus structure or a tubular structure cannot be formed on demand when required. Indeed, the formation of tubular structures in vitro is completely random and to date a method for forming tubular structures with any degree of control or reproducibility has not been described.

Finally, current three-dimensional culture systems do not restrain cell movement, which makes time lapse observation of organoid formation a very complicated task that requires highly efficient tracking systems.

Therefore, there is a need for a cell culture system that allows the controlled formation of organoids or three-dimensional multicellular assemblies suitable for use in high-throughput experimental studies, and in particular a cell culture system provides three-dimensional multicellular tubular structures.

### SUMMARY

The present invention provides devices and methods that allow the controlled formation of organized three-dimensional multicellular assemblies suitable for use in high-throughput experimental studies.

Accordingly, in a first aspect, the present invention provides a device comprising:

a support defining a surface;

at least one two-dimensional adhesive pattern on said surface, and

at least one three-dimensional multicellular assembly immobilised on said two-dimensional adhesive pattern, wherein said three-dimensional multicellular assembly has an organised structure with a normalised polarity.

The device according to the present invention comprises a three-dimensional multicellular assembly that is immobilised on the adhesive pattern, not embedded in gel, which facilitates imaging of the assembly (FIG. 1). Besides, the adhesive pattern is two-dimensional, meaning that it does not consist in a well whose walls would assist the three-dimensional assembly of the cells. To the contrary, the three-dimensional multicellular assemblies are grown on the adhesive pattern, being only in contact with the support via this two-dimensional surface. Further, the resultant assembly is immobilised at a predetermined position on the support, ie. on the adhesive pattern, which further facilitates imaging and allows time lapse observation. Moreover, the three-dimensional multicellular assembly has an organised structure, which means the structure exhibits a normalised polarity and a more physiologically relevant architecture than has previously been achieved in organoid cultures.

The three-dimensional multicellular assembly may be any assembly or arrangement known in the art. In one particular embodiment, the three-dimensional multicellular assembly comprises at least one lumen. In another embodiment, said three-dimensional assembly is a structure selected from the group comprising an acini and a tube.

It will be understood that the specific characteristics of the adhesive pattern on the support will be dependent on the specific characteristics of the three-dimensional assembly and/or the characteristics of the cells comprising the three-dimensional assembly.

In one embodiment, said adhesive pattern is adapted to control initial cell spreading of the three-dimensional multicellular assembly. Cell spreading may be controlled by any means known in the art. In one embodiment, control of initial cell spreading is by a means selected from the group comprising topographically and chemically, or by a combination thereof. In one embodiment, topographical control of initial cell spreading comprises forming an adhesive pattern from a concave shape in the surface of said support. In another embodiment, chemical control of initial cell spreading comprises forming an adhesive pattern from a cytophobic and/or cytophilic material. In another embodiment, the adhesive pattern is adapted to accommodate a single or few cells. In a further embodiment, the adhesive pattern is adapted to influence the polarity of cells. In a yet further embodiment, the adhesive pattern is adapted to accommodate a single or few cells.

In one embodiment, the adhesive pattern is approximately a shape selected from the group comprising a disc, a crossbow, an H, a Y, a rectangle, a ring and a S, or any combination thereof. In a particular embodiment, said adhesive pattern is adapted to promote the formation of a tube.

Accordingly, the present invention also provides devices and methods that allow the controlled formation of three-dimensional tubular structures. Therefore, in a second aspect, the present invention provides a device for forming a three-dimensional multicellular tubular structure comprising:

a support defining a surface; and

at least one two-dimensional adhesive pattern on said surface, wherein said adhesive pattern is adapted to form a tubular structure. In one embodiment, the adhesive pattern has a length to width ratio of between about 5:1 and about 15:1. In another embodiment, the adhesive pattern has a length to width ratio of 10:1.

In a fourth aspect, the present invention provides a method of forming a three-dimensional multicellular tubular structure comprising:
(i) providing a support comprising at least one two-dimensional adhesive pattern, wherein said adhesive pattern is adapted to form a tubular structure;
(ii) seeding said support with cells; and
(iii) culturing said cells under conditions and for a sufficient period of time to obtain a three-dimensional multicellular tubular structure on the two-dimensional adhesive pattern.

The ability to promote the formation of a three-dimensional multicellular assembly with an organised structure and specific characteristics allows the construction of a device comprising multiple three-dimensional multicellular assemblies that are substantially similar, which is useful in conducting high-throughput experimental studies.

Accordingly, in a fifth aspect, the present invention provides a device comprising:

a support defining a surface;

at least two two-dimensional adhesive patterns on said surface; and

at least two three-dimensional multicellular assemblies immobilised on said two-dimensional adhesive patterns, wherein said three-dimensional multicellular assemblies have an organised structure with a normalised polarity and wherein said three-dimensional multicellular assemblies have substantially the same structure.

It will be appreciated that the present invention also provides methods of forming a three-dimensional multicellular assembly with an organised structure. In one embodiment, there is provided a method of forming a three-dimensional multicellular assembly comprising:
(i) providing a support comprising at least one two-dimensional adhesive pattern according to the present invention;
(ii) seeding said support with cells; and
(iii) culturing said cells under conditions and for a sufficient period of time to obtain a three-dimensional multicellular assembly on said two-dimensional adhesive pattern, wherein said three-dimensional multicellular assembly has an organised structure.

Accordingly, in a sixth aspect, the present invention provides a method of forming a three-dimensional multicellular assembly comprising:
(i) providing a support comprising at least one two-dimensional adhesive pattern;
(ii) seeding said support with cells; and
(iii) culturing said cells under conditions and for a sufficient period of time to obtain a three-dimensional multicellular assembly on said two-dimensional adhesive pattern, wherein said three-dimensional multicellular assembly has an organised structure with a normalised polarity.

In one embodiment, step (iii) comprises overlaying the cells with a culture medium. In a particular embodiment, the culture medium comprises extracellular matrix proteins.

In another embodiment, step (ii) further comprises incubating the support and cells for a sufficient period of time to allow the cells to adhere to the support. In yet another embodiment, step (ii) further comprises the step of washing the support to remove cells not adhered to the support.

In a further embodiment, the method further comprises the step of preparing a support comprising at least one adhesive pattern, wherein said preparation comprises:

(a) activating the surface of a glass support;
(b) coating the active surface with a cytophobic polymer, and
(c) printing a two-dimensional adhesive pattern onto the coated surface.

It will be understood that the three-dimensional multicellular assemblies may be formed from any cell. In one embodiment, the cells are selected from the group comprising pluripotent stem cells, epithelial cells, or epithelial-like cells and epithelial-derived cells. In a particular embodiment, the cells are Madin-Darby Canine Kidney (MDCK) or Caco-2 cells.

In one embodiment, the present invention also relates to a three-dimensional multicellular assembly produced according to the methods and devices described herein.

In another embodiment, the present invention relates to a three-dimensional culture system comprising:
(i) a support comprising at least one two-dimensional adhesive pattern according to the present invention; and
(ii) instructions for forming a three-dimensional multicellular assembly according to the present invention.

In a further embodiment, the present invention relates to a method of defining an average cell comprising:
(i) providing a support comprising at least one two-dimensional adhesive pattern according to the invention;
(ii) seeding said support with cells; and
(iii) forming organised three-dimensional multicellular assemblies with a normalised polarity on said two-dimensional adhesive pattern according to the methods of the present invention, wherein said three-dimensional multicellular assemblies have substantially the same structure, shape and size; and
(iv) imaging said three-dimensional multicellular assemblies and defining an average cell by averaging data obtained from images of said three-dimensional assemblies.

### DESCRIPTION OF THE FIGURES

FIG. 1: Schematic representation of three-dimensional culture systems. A. Previous methods: Cells are seeded on a thick gel and resultant cells are embedded in the gel; and B. Method according to one embodiment of the present invention: Cells are seeded on a micropatterned adhesive support without any gel.

FIG. 2: Schematic of an acinus/tube, follicle and spheroid showing normalised apicobasal polarity in the acinus/tube and follicle and the absence of normalised apicobasal polarity in the spheroid.

FIG. 3: Schematic examples of adhesive patterns according to embodiments of the present invention.

FIG. 4: MDCK three-dimensional 2-cell assemblies formed on a collagen-I H-shaped micropatterned adhesive support after 24 hours. Polarity markers are immunofluorescently labelled in green (apical) and red (baso-lateral), nuclei are stained in blue (Hoechst).

FIG. 5: Normalised images of MDCK three-dimensional 2-cell assemblies formed on a collagen-I H-shaped micropatterned adhesive support after 24 hours. Polarity markers are immunofluorescently labelled in green (apical) and red (baso-lateral), nuclei are stained in blue (Hoechst).

FIG. 6: An acinus formed from MDCK cells on a laminin Y-shaped micropatterned adhesive support immunofluorescently labelled for polarity markers (apical=green; baso-lateral=red; nuclei=blue).

FIG. 7: Three-dimensional multicellular assemblies on micropatterned adhesive support formed from MDCK cells. A. An acinus formed on a laminin Y-shaped micropattern after 3 days. B. A tube on a collagen-I tubular micropatterned adhesive support formed from multiple H-shaped micropatterns after 4 days. Polarity markers are immunofluorescently labelled in green (apical) and red (baso-lateral), nuclei are stained in blue (Hoechst).

FIG. 8: Three-dimensional multicellular assemblies formed on a collagen-I disc-shaped micropatterned adhesive support from Caco-2 cells after 5 days. A. A follicle overlaid with complete culture medium. B. Acini overlaid with complete medium supplemented with 2% Matrigel™ and 5% Matrigel™. Polarity markers are immunofluorescently labelled in red (apical) and green (baso-lateral), nuclei are stained in blue (Hoechst).

FIG. 9: Normalised localisation of nuclei and actin in three-dimensional MDCK 2-cell assemblies formed on a collagen-I H-shaped micropatterned adhesive support.

FIG. 10: MDCK cells on Y-shaped micropatterns 5 hours after seeding. Top: Cell on a collagen-I micropattern. Bottom: Cell on a laminin micropattern.

FIG. 11: Characterisation of 3D acini size distribution on a Starter's CYTOOchip. MDCKII acini were formed on a Starter CYTOOchip for 72 hours in MEM medium+2% FCS supplemented with 2.5% Matrigel. After fixing, structures were stained for DNA, F-actin, gp135 and imaged. 3D acini size (μm²) distribution is represented in the box-plot graphs on each of the pattern shapes and size: median values are shown by the horizontal bar within each box, boxes show 25th and 75th percentiles, whiskers show the spread of the data.

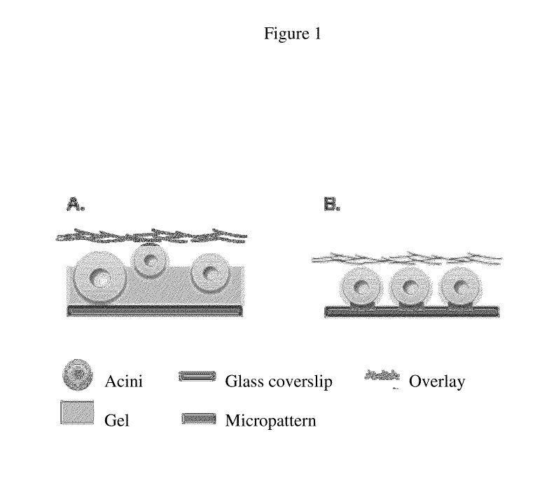

NP=non-patterned; D=disc pattern; C=crossbow pattern; H=H pattern; Y=Y pattern
L=large (1700 μm²); M=medium (1100 μm²); S=small (700 μm²) n=number of acini.

### DETAILED DESCRIPTION OF THE PREFERRED EMBODIMENTS

Before describing the present invention in detail, it is to be understood that this invention is not limited to particularly exemplified methods and may, of course, vary. It is also to be understood that the terminology used herein is for the purpose of describing particular embodiments of the invention only, and is not intended to be limiting which will be limited only by the appended claims.

All publications, patents and patent applications cited herein, whether supra or infra, are hereby incorporated by reference in their entirety. However, publications mentioned herein are cited for the purpose of describing and disclosing the protocols and reagents which are reported in the publications and which might be used in connection with the invention. Nothing herein is to be construed as an admission that the invention is not entitled to antedate such disclosure by virtue of prior invention.

The practice of the present invention will employ, unless otherwise indicated, conventional techniques of cell culture, cell biology and microfabrication, which are within the skill of the art. Such techniques are described in the literature. See, for example, Coligan et al., 1999 “Current protocols in Protein Science” Volume I and II (John Wiley & Sons Inc.); Ross et al., 1995 “Histology: Text and Atlas”, 3rd Ed., (Williams & Wilkins); Kruse & Patterson (eds.) 1977 “Tissue Culture” (Academic Press); and Alberts et al. 2000 “Molecular Biology of the Cell” (Garland Science).

It must be noted that, as used herein and in the appended claims, the singular forms “a”, “an”, and “the” include plural reference unless the context clearly dictates otherwise. Thus, for example, a reference to “a cell” includes a plurality of such cells, and a reference to “an adhesive pattern” is a reference to one or more adhesive patterns, and so forth.

Unless defined otherwise, all technical and scientific terms used herein have the same meanings as commonly understood by one of ordinary skill in the art to which this invention belongs.

Although any materials and methods similar or equivalent to those described herein can be used to practice or test the present invention, the preferred materials and methods are now described.

In its broadest sense, the present invention generally relates to a device comprising a three-dimensional multicellular assembly. The term “three-dimensional multicellular assembly”, “three-dimensional multicellular assemblies”, or grammatical equivalents thereof, refers to any arrangement comprising two or more cells.

The three-dimensional multicellular assembly of the present invention has an organised structure. The term “organised structure”, as used herein, indicates that the arrangement of the cells that constitute the assembly is coordinated. Previously described three-dimensional assemblies comprised a random collection of cells having a random placement and orientation within the assembly. In contrast, the cells of the three-dimensional assemblies according to the present invention are not arranged randomly, but are coordinated with regard to their position and orientation in relation to each other and the assembly. In other words, the three-dimensional multicellular assembly of the present invention has a “normalised polarity”. The concept of cell and tissue polarity and normalised polarity is well known to those skilled in the art (as described, for example, in Bryant and Mostov, Nat Rev Mol Cell Biol. 2008 November, 9(11): 887-901). However, briefly, cell polarity is a fundamental feature of almost all eukaryotic cells. Most cells have a single, clear axis of asymmetry, i.e. a “front” and a “back”. In situ, the polarity of a cell must be coordinated in space and time in order for individual cells to form a tissue. For example, epithelial cells feature distinct “apical” and “basolateral” surfaces. Within tissues, the apical surfaces of all cells face the lumen, while the basolateral surfaces of all cells face other cells and the extracellular matrix. Accordingly, the term “normalised polarity”, as used herein, indicates that substantially all the cells in the three-dimensional multicellular assembly have an axis of asymmetry and that substantially all cells in the assembly are orientated according to their axis of asymmetry in a physiologically relevant manner.

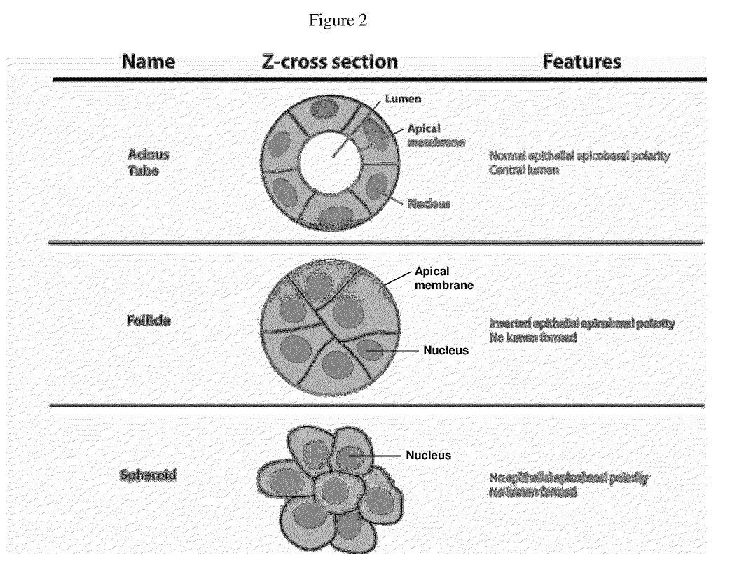

A person skilled in the art would appreciate that the particular organisation and orientation of the cells will be dependent on the type of assembly formed. In a particular embodiment, the three-dimensional multicellular assembly has a “normalised apicobasal polarity”. The term “normalised apicobasal polarity” or “normalised apical-basal polarity”, indicates that substantially all the cells of the assembly comprise an “apical” surface and a “basolateral” surface and that substantially all the cells of the assembly are orientated according to this axis of asymmetry in a physiologically relevant manner. For example, substantially all the cells in an acinus will be orientated with their apical pole facing towards a closed inner cavity or lumen and their basolateral surface facing the culture medium. However, in a follicle, substantially all the cells will be orientated with their apical pole facing the culture medium. These formations are in contrast to the three-dimensional assemblies described in the prior art, which generally formed spheroids that are without normalised apicobasal polarity (see FIG. 2).

The polarity of cells within a three-dimensional multicellular assembly can be readily determined by the person skilled in the art using routine imaging techniques. For example, a three-dimensional multicellular assembly on a micropatterned adhesive support may be fixed using a fixative reagent such as paraformaldehyde or methanol. The fixed assembly is then stained using immunofluorescence techniques that allow the visualization of different compartments such as nuclei, microfilaments, micrtubules, apical and basolateral membranes. For example, Gp135, a protein specifically localized at the apical pole of the cells, can be labelled and the orientation of the cells visualized. Other markers used in immunofluorescence include phalloidin (apical), β-catenin (basolateral), Hoechst (nucleus/DNA) and DAPI (nucleus/DNA). The micropatterned adhesive supports with the fixed, stained assemblies can then be mounted on glass slides with a mounting medium such as ProLong® Gold (Invitrogen, 5791 Van Allen Way, Carlsbad, Calif. 92008, U.S.A.). Fluoromountm (Sigma-Aldrich, 3050 Spruce Street, St. Louis, Mo. 63103, U.S.A.) or Mowiol® 4-88 (Polysciences Europe GmbH. Htandelsstrasse 3 D-69214 Eppelheim, Germany). Slides can then be analyzed using confocal or wide-field microscopes to acquire images of the different structures in the assemblies.

It is to be noted that whereas staining may be necessary for certain imaging techniques, three-dimensional assemblies according to the invention may also be used for in vivo imaging experiments that do not require staining.

The skilled person is able to determine if staining is necessary and, if appropriate, to define a suitable stain.

It will be understood that the three-dimensional assemblies of the present invention may form any structure known in the art. Preferably, three-dimensional multicellular assemblies have a structure similar to that of the in vivo tissue from which the cells originate. Preferred, three-dimensional structures are selected from any of the following: a follicle, an acinus or a tube.

In a particular embodiment, the cells of the three-dimensional multicellular assembly enclose a lumen. The term “lumen”, as used herein, refers to a space or cavity enclosed by cells and defined by cell-cell junctions, wherein said space or cavity is substantially free of cells and isolated from the culture medium. Typically, a lumen will have a physiologically different composition from the culture medium as a result of cell activity. Generally, an assembly will comprise a single lumen; however, in larger assemblies multiple lumens may be present. In particular embodiments of the present invention the three-dimensional multicellular assembly is an acinus or a tube. The term “acinus” or “acini”, as used herein, refers to an approximately spherical, multicellular assembly that comprises at least one lumen. The terms “tube” and “tubular”, as used herein, refers to an elongated, multicellular assembly that comprises at least one lumen.

It will be appreciated that the cells that constitute a three-dimensional multicellular assembly of the present invention may be any cell capable of forming three-dimensional multicellular assembly and such cells are known to those skilled in the art. Further, the selection of cell type will be dependent on the purpose for which the three-dimensional multicellular assembly is required. Preferably, the cell is a eukaryotic cell and more preferably a mammalian cell.

As used herein the term “mammalian cell” refers to cells derived from a mammal, or mammalian tumour, including human cells. All mammalian animals are composed of groups of cells that specialise in performing a particular function or “tissues”. Cells that are particularly suited to the methods and devices of the present invention are derived from tissues including epithelium (epithelia) tissue, connective tissue, nerve tissue and muscle tissue. All of these tissues comprise cells that have phenotypic characteristics in common across species. For example, epithelia from all mammalian species generally comprise a single layer of cells held together by occluding junctions called tight junctions. More importantly, all cells within epithelia from any mammalian species have similar growth characteristics. Alternatively, the cells may be immature cells with the ability to differentiate into multiple cell types, for example totipotent, pluripotent or multipotent stem cells. The cells may be derived from healthy or diseased tissue. Preferred cells are pluripotent stem cells, epithelial cells, or epithelial-like cells and epithelial-derived cells.

Epithelial cells include cells derived from the skin, lung, intestinal epithelial, colon epithelial, testes, breast, prostate, brain, kidney, ovary and thymus. The term “epithelial-like cells”, refers to cells resembling, characteristic of, having the form or appearance of epithelial cells. The term “epithelial-derived cells” refers to populations of cells that have originated from an epithelial cell, for example epithelial-derived cell lines and cancers. Accordingly, examples of epithelial-derived cells include cell lines and tumour cells derived from skin cells, lung cells, intestinal epithelial cells, colon epithelial cells, testes cells, breast cells, prostate cells, brain cells, kidney cells, ovary cells and thymus cells. Preferred cell lines are selected from any one of the following: MDCK, Caco-2, RPE-1, CHO, BSC and MCFIOA.

Preferably, cells, in particular stem cells, are not obtained by a method requiring destruction of human embryos.

Traditionally, once seeded, cells begin to grow and spread on the surface on which they are placed; however, the three-dimensional assembly of the present invention is immobilised on an adhesive pattern. The term “adhesive pattern” as used herein, refers to any area that defines a surface to which cells are capable of adhering. Without wishing to be bound by any particular theory, it is believed that by containing initial cell spreading and cell adhesion the formation of an organised three-dimensional assembly is promoted. Accordingly, in one embodiment, the device comprises an adhesive pattern that controls initial cell spreading. Cell spreading may be controlled by any means known in the art. For example, spreading of the three-dimensional multicellular structure may be controlled topographically or chemically, or by a combination thereof. For example, initial cell spreading can be controlled topographically by forming an adhesive pattern from a concave shape in the surface of a support. Initial cell spreading can also be controlled chemically, for example, by positively forming an adhesive pattern with a cytophilic material. Preferred cytophilic materials are selected from any one of the following: laminin proteins, collagen type I (collagen-I) proteins, BME-extracts, collagen type-IV proteins, fibronectin, anti-cadherin Fab antibody fragments and anti-integrin blocking antibodies. Alternatively, initial cell spreading can be controlled chemically by negatively forming an adhesive pattern with a cytophobic material. Preferred cytophobic materials are derivatives of oligo or poly(ethylene)glycol like poly(ethylene glycol)-poly-L-lysine (PEG-PLL), polyethylene oxide, poly(vinyl acetate), poly(2-hydroxyethyl methacrylate), polyacrylamide, poly(N-vinyl-2-pyrrolidone), poly(N-isopropylacrylamide), silicons like PDMS (polydimethylsiloxane), silanes (perfluorinated silanes in particular), anionic polymers, phosphorylcholine polymers, albumin, casein, hyaluronic acid, liposaccharides, glycoproteins, phospholipids or a mix of these compounds.

Control of initial cell spreading may also be aided by employing an adhesive pattern adapted to accommodate a single or few cells. The term “accommodate a single or few cells”, as used herein, indicates that the size of the adhesive pattern is such that there is only sufficient area in the pattern for one or very few cells to adhere. The term “few cells”, as used herein, will typically indicate less than 10 cells, preferably less than five cells. Examples of such patterns are well known in the art and have been described elsewhere, see, for example, Bornens et al. WO 2005/026313.

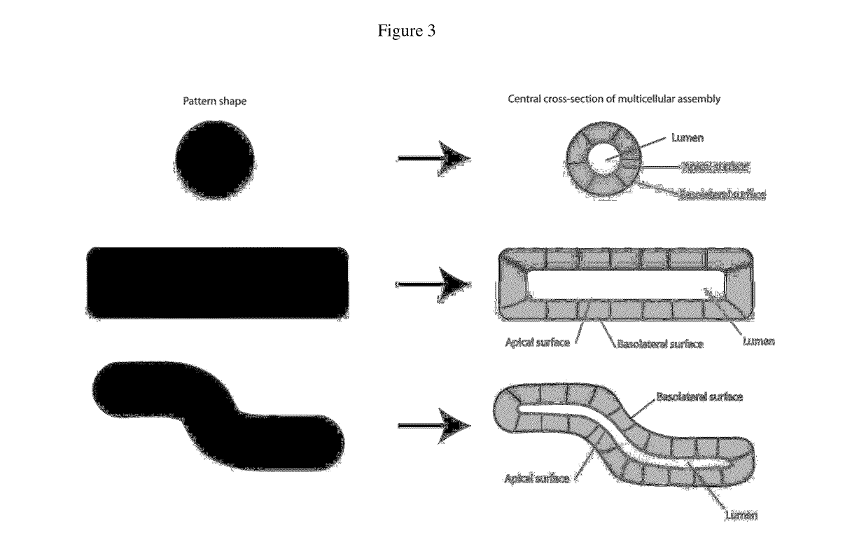

It will be understood that the specific characteristics of the adhesive pattern on the support will be dependent on the specific characteristics desired in the three-dimensional assembly and/or the characteristics of the cells being used to construct the three-dimensional assembly. For example, a larger pattern will result in a larger three-dimensional assembly. Additionally, an adhesive pattern adapted to promote the formation of a tube may comprise an elongated shape, while an adhesive pattern adapted to promote the formation of an acinus or a follicle will not. In one embodiment, said adhesive pattern is approximately the shape of a disc, a crossbow, an H and a Y, or any combination thereof. In another embodiment, said adhesive pattern is approximately a shape selected from the group comprising a rectangle, a ring and a S. The adhesive pattern may comprise a single shape or comprise multiple shapes. Additionally, the adhesive pattern may be constructed to influence the polarity of cells. Examples of such patterns are well known in the art and have been described elsewhere, see, for example, Bornens et al. WO 2005/026313. Examples of specific adhesive patterns are discussed infra and are shown in FIG. 3. Preferred adhesive patterns are selected from any one of the following: a disc, a crossbow, an H, a Y, a rectangle, a ring or a S.

As discussed above, the present invention also relates to devices comprising tubular structures and methods for the formation of tubular structures. Adhesive patterns adapted to form tubular structures will generally comprise an elongated shape; however, tubular structures may also be formed from shapes other than rectilinear shapes, for example rings.

Preferred adhesive patterns adapted to form a tubular structure have a length to width ratio of about between about 2:1 and about 20:1, more preferably between about 5:1 and about 15:1 and even more preferably about 10:1. Preferably, the adhesive pattern adapted to form a tubular structure has a length of greater than about 100 μm, more preferably between about 100 and about 300 μm, and even more preferably about between about 200 and about 300 μm. The term “length”, as used herein in reference to a tubular structure, refers to the length of the lumen forming structure not the length of the shape of the structure. For example, the length of a ring forming a tubular structure will be the circumference of the ring shape, not the diameter of the ring shape.

Accordingly, the characteristics of the three-dimensional assembly are in part controlled or predetermined by the arrangement of the adhesive pattern. In other words, the present invention provides a method of creating a three-dimensional multicellular assembly having a predetermined structure, shape and size. The term “predetermined structure, shape and size”, as used herein, refers to a three-dimensional multicellular assembly wherein the structure, shape and size of the three-dimensional multicellular assembly is defined prior to the commencement of formation (ie. prior to seeding) and the resultant three-dimensional multicellular assembly has substantially the desired structure, shape and size.

The “structure” of a three-dimensional multicellular assembly, refers to the type of assembly formed, for example, a follicle, acinus or tube. As discussed supra, the three-dimensional assemblies of the present invention may form any structure known in the art. Examples of structures relevant to the present invention, including the characteristics of and methods of identifying and forming such structures are discussed elsewhere herein.

The “shape” of a three-dimensional multicellular assembly, refers to the spatial form or contour of the three-dimensional multicellular assembly. The three-dimensional multicellular assembly may be any shape known in the art, including a sphere, a rectangle, a ring or a S. Further, the shape of a three-dimensional multicellular assembly can be readily determined by the skilled person using routine techniques. For example, three-dimensional multicellular assemblies may be fixed and stained using a classical immunofluorescence protocol and then imaged through a classical reflected epifluorescence microscope, or maintained alive and observed in phase contrast throughout the time using a videomicroscope. The resulting images can be then analysed to measure different shape factors on the multicellular assemblies. As mentioned above, staining may not be required, depending on the imaging technique.

The “size” of a three-dimensional multicellular assembly, refers to the dimensions of the three-dimensional multicellular assembly, ie. the length, width and depth of the assembly, or the number of cells that comprise the three-dimensional multicellular assembly. The dimensions of a three-dimensional multicellular assembly can be readily determined by a person skilled in the art using routine techniques, including the imaging techniques described supra. Alternatively, for very small three-dimensional multicellular assemblies comprising very few cells, it may be more convenient to measure the size of the assembly by the number of cells the assembly comprises. The number of cells in a three-dimensional multicellular assembly can be readily determined by the skilled person using routine techniques. For example, the nuclei of cells constituting the multicellular assemblies can be stained with Hoechst, then imaged and simply counted with an automatic program (e.g. an ImageJ macro). The number of nuclei per multicellular assembly corresponds to the number of cells per multicellular assembly.

It will be understood that the specific size of a particular three-dimensional multicellular assembly formed according to the present invention will be dependent on the requirements of the user and may be limited by the type of cell used to construct the three-dimensional multicellular assembly. Methodologies for determining what size is most suitable for a particular cell type are know in the art and could be readily determined by a person skilled in the art through routine experimentation. For example, micropatterned adhesive supports comprising micropatterns of various sizes are commercially available and could be used to determine what dimensions are most suited to forming a three-dimensional multicellular assembly from a particular cell type (eg. CYTOO's Starter CYTOOchip, CYTOO S. A. 7, parvis Louis Néel, BHT 52, BP50, 38040 Grenoble cedex 9, France).

An advantage of forming three-dimensional multicellular assemblies with a predetermined structure, shape and size, is that this allows the construction of a device comprising multiple organised three-dimensional multicellular assemblies that have substantially the same structure, shape and size, which can be used in high-throughput experimental studies.

The term “substantially the same structure, shape and size”, as used herein, indicates that the three-dimensional multicellular assemblies have a similar structure, shape and size and that these features do not differ significantly between assemblies.

Accordingly, as used herein, “substantially the same structure”, has the meaning that the assemblies are composed of cells having substantially the same shape and size in substantially the same orientation and position. For example, the device may comprise an array of acinus each comprising a single lumen, wherein substantially all the cells are orientated such that the apical poles of the cells face the lumens of the assemblies.

As used herein “substantially the same shape”, has the meaning that the overall spatial form or contour of the three-dimensional multicellular assemblies does not differ significantly between said assemblies. For example, two assemblies of an approximately rectangular shape will have substantially the same shape; however, one assembly of an approximately spherical shape and one assembly of an approximately rectangular in shape will not be substantially the same shape.

As used herein “substantially the same size”, has the meaning that the dimensions (ie. the length, width and depth) of the three-dimensional multicellular assemblies or the number of cells comprising the three-dimensional multicellular assemblies do not differ significantly between said assemblies. Typically said difference is not more than 50%, preferably not more than 25%, most preferably not more than 10%.

Methods for determining the structure, shape and size of multiple three-dimensional multicellular assemblies are known to those skilled in the art and are discussed supra and infra.

It will be appreciated that devices according to the present invention may comprise an array of organised three-dimensional multicellular assemblies that have substantially the same structure, shape and size. Alternatively, the device may comprise an array of organised three-dimensional multicellular assemblies divided into sections comprising different organised three-dimensional multicellular assemblies, but wherein the three-dimensional multicellular assemblies within the sections have substantially the same structure, shape and size. For example, the device may comprise an array of acini and an array of tubes, or alternatively an array of acini of one size and an array of acini of a different size.

Further, the ability to create organised three-dimensional multicellular assemblies that have substantially the same structure, shape and size allows the definition of an “average cell” or “reference cell”. An average cell is created by averaging the data obtained from a number of similar cells within several similar three-dimensional multicellular assemblies. Examples of data include structural information relating to the cell, for example, the size and shape of the cell and the position, size and shape of cellular compartments such as the primary cilium, the apical membrane, the centrosome, the nucleus, microfilaments, microtubules, the Golgi stacks, and mitochondria. While data from two cells is sufficient to define an “average cell”, the accuracy of the resultant “reference cell” increases with the number of cells from which data is collected. Preferably data from at least two cells is used, more preferably at least six cells and even more preferably at least 10 cells.

Methods for collecting such data and building an average cell are known to those skilled in the art and described, for example, in Théry et al., Cell Motil Cytoskeleton 2006, 63(6):341-55. However, briefly, three dimensional cultures may be treated with the fixative reagent 4% PFA and immunostaining performed for different cell compartments (such as the primary cilium, the apical membrane, the centrosome, the nucleus, microfilaments, microtubules, the Golgi stacks, mitochondria). Images can then be acquired using confocal or wide-field deconvolution microscopy techniques. An array of at least 10 individual images of representative cells is created from the same sample. For each stained organelle a stack of images can be created. Translational alignment of different images can be performed using the centroid of one of the organelles (typically the nucleus) as a spatial reference. Then, a projection of each stack of images can be performed by summing individually the signal of each pixel in all of the stacks. The image resulting from such projection is then combined with the resulting image of each organelle using different color codes. Intensity measures, organelle distribution and other derived parameters can then be used for analysis.

It will be appreciated that the present invention also contemplates methods of forming a three-dimensional multicellular assembly having an organised structure. Accordingly, in one embodiment, a support having at least one adhesive pattern substantially as described above may be seeded with cells. Once seeded onto the support, the cells are then cultured under conditions and for a sufficient period of time to obtain a three-dimensional multicellular assembly. Suitable methods and materials for culturing cells are known to those skilled in the art and are described infra.

It will be appreciated that culture conditions and time frames will, of course, vary depending on the type of cell being grown and the size of the assembly being formed. In one embodiment, the cells are cultured for at least about 24 hours, more preferably at least about 48 hours. In one particular embodiment, culturing the cells comprises overlaying said cells with a culture medium. The term “overlaying”, as used herein, involves applying a sufficient volume of culture medium to the cells such that the cells are fully covered. In one embodiment, the culture medium comprises extracellular matrix proteins. Suitable culture media and extracellular matrix proteins appropriate for use in the present invention are known to those skilled in the art. In a preferred embodiment the overlay comprises a commercially produced culture medium such as Matrigel™ (BD, 1 Becton Drive, Franklin Lakes, N.J. U.S.A.). In one embodiment, the overlay comprises between about 2% and about 100% Matrigel™, more preferably between about 2% and about 20% Matrigel™, even more preferably between about 2% and about 5% Matrigel™ and even more preferably about 2% Matrigel™.

In one embodiment, prior to culturing the cells, the method comprises a step of incubating said support and cells for a sufficient period of time to allow the cells to adhere to the support. The period time required for cells to adhere to the support will, of course, vary depending on the cell type and other conditions such as the nature of the adhesive pattern. In another embodiment, the method comprises a step of washing the support to remove cells that are not adhered to the support. Methods and solutions useful in the washing process are known by those skilled in the art and discussed infra.

Additionally, the present invention also contemplates methods of preparing a support comprising adhesive patterns, as well as kits, three-dimensional culture systems, methods of screening and the like. In one embodiment, the method of the present invention further comprises the step of preparing a support comprising at least one adhesive pattern, wherein the preparation comprises: (a) activating the surface of a glass support; (b) coating the active surface with a cytophobic polymer; and (c) printing an adhesive pattern onto the coated surface. In another embodiment, there is provided a three-dimensional culture system comprising: (i) a support comprising at least one adhesive pattern; and (ii) instructions for forming a three-dimensional multicellular assembly according to the present invention.

As described above, the methods and devices of the present invention provide multiple three-dimensional multicellular assemblies that are substantially similar, which may be useful in conducting high-throughput experimental studies. In one embodiment, the present invention provides a method of screening for substance toxicity, absorption or therapeutic application comprising contacting three-dimensional multicellular assemblies formed according to the methods described above with a substance; determining any phenotypic or metabolic change in the cells of the assembly that results from contact with said substance; and correlating said change with cellular toxicity, cellular absorption or therapeutic effect.

Additionally, the process of cell seeding, spreading and organoid formation can be followed by live-cell videomicroscopy using incubation chambers specially suited for the supports. These cultures can be treated (before, after or during lumen formation) with different reagents to evaluate their effect on epithelial structure, permeability, lumen formation efficiency or cell migration. Studies are performed on live cells, or cells can be fixed and stained to study the localization of different cellular markers.

### EXAMPLES

### Example 1

### Preparing the Patterned Support

Glass supports were oxidized in a plasma chamber to activate the surface, and coated with poly(ethylene glycol)-poly-L-lysine (PEG-PLL). Patterns of different shapes were then printed by photolithography. A sufficient volume of laminin or collagen-I at 20 μg/mL in phosphate buffered saline (PBS) was then applied to completely cover the support before incubation for 2 hours at room temperature or overnight at 4° C. After incubation, the solution was replaced with PBS and then supports were washed twice with 10 ml of PBS for 1 hour. The PBS solution was then aspirated and the supports were left to dry. The supports were either immediately used or stored at 4° C. for 24-48 hours.

### Example 2

### Cell Deposition

MDCK cells (MDCK (NBL-2) ATCC CCL-34™) obtained from American Type Culture Collection (ATCC; Manassas, Va. U.S.A.) were washed twice with PBS. The first washing was rapid whereas the second washing consisted of incubating the cells in PBS for 20-30 minutes at room temperature. After that, cells were detached from their flask by the addition of trypsin-EDTA and incubated for 5 minutes at 37° C. Complete culture medium was added to the flask and collected cells were centrifuged for 4 minutes at 1400 rpm. The supernatant was removed and the cells were resuspended in complete culture medium comprising Minimum Essential Medium with Glutamax-I, Earles (Invitrogen, 5791 Van Allen Way, Carlsbad, Calif. U.S.A.) supplemented with 10% fetal bovine serum and 1% penicillin-streptomycin. The cell solution was then applied to the micropatterned adhesive support at a final density of about 10,000-20,000 cells per cm². The cells and micropatterned adhesive support were then incubated at room temperature for about 30-40 minutes or until the majority of cells were attached to the micropatterns. Non-attached cells were removed with a flow of medium added to one side of the support and aspirated on the other. The cell-seeded support was then placed in a cell incubator (37° C., 5% CO2) for a further 3 hours to allow cells to completely adhere to the support.

### Example 3

### Overlaying Cells with a Culture Medium Comprising Extracellular Matrix proteins

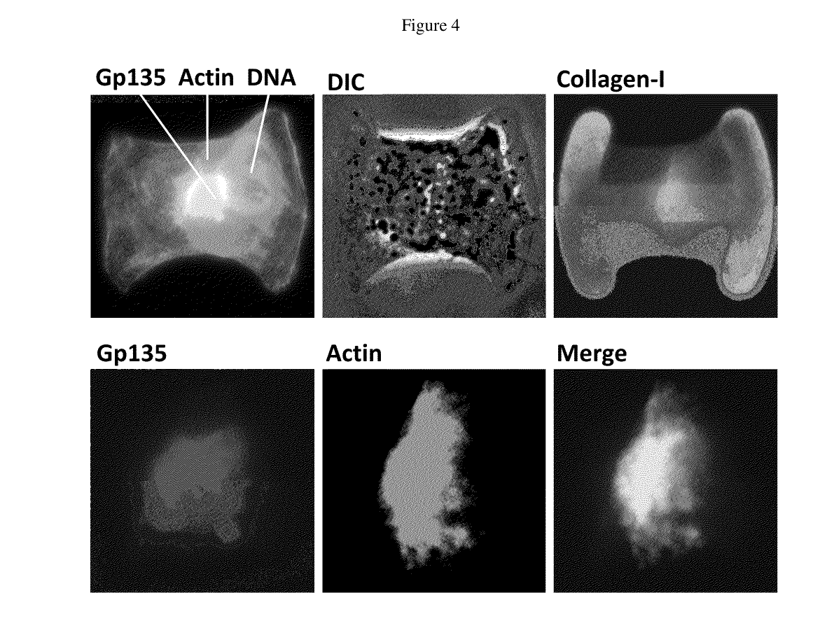

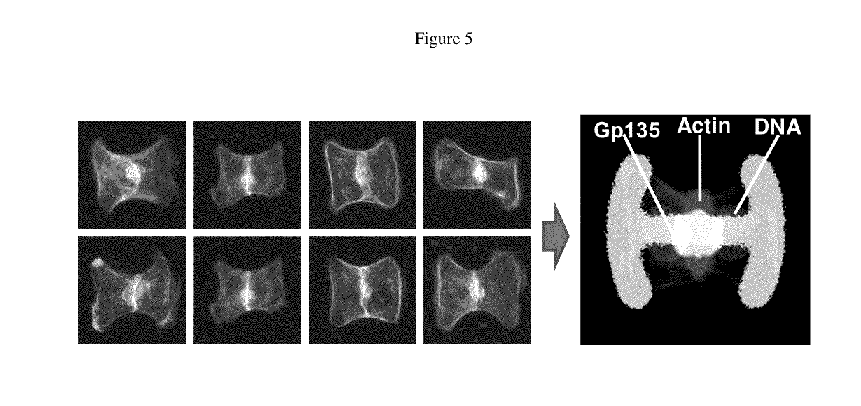

A solution of 2% Matrigel™ (BD, 1 Becton Drive, Franklin Lakes, N.J. U.S.A) was prepared in cold complete culture medium and then warmed to 37° C. Three hours after seeding, the cell-seeded support media was replaced with the diluted Matrigel™ MDCK cells seeded on collagen-I H-shaped micropatterned adhesive supports formed multicellular aggregates and lumen formation was evident after 24 hours (FIGS. 4 and 5). The aggregates continued to grow until cellular structures were unable to be supported and detached from the support (approximately day 8, not shown).

### Example 4

### Formation of Three-Dimensional Structures

MDCK cells were prepared and seeded onto micropatterned adhesive supports as described in Examples 1-3 and allowed to form three-dimensional aggregates. The Matrigel™ solution was replaced every 48 hours.

Once three-dimensional aggregates were formed, aggregates were fixed on the support using paraformaldehyde 4% for 20 minutes and permeabilized with Triton-X100 0.1%-PBS solution. The apical membrane was stained with a primary antibody anti-GP135 and a secondary antibody anti-mouse IgG-DyLight 488 (Thermo Fisher Scientific Inc., 3747 North Meridian Road, Rockford, Ill. 61101, U.S.A). The baso-lateral membrane was stained with a primary antibody anti-β-catenin and a secondary antibody anti-rabbit IgG-Cy3. The nucleus (DNA) was stained with Hoechst. After fixing, supports were mounted on glass slides with ProLong® Gold (Invitrogen, 5791 Van Allen Way, Carlsbad, Calif. 92008, U.S.A.). Slides were analyzed using confocal or wide-field microscopes to acquire images of the different structures in the acini.

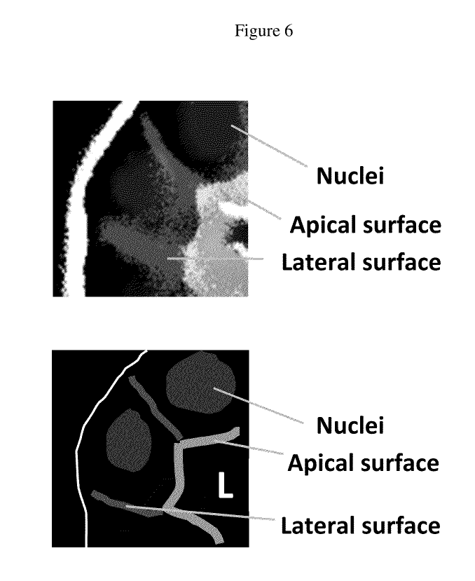

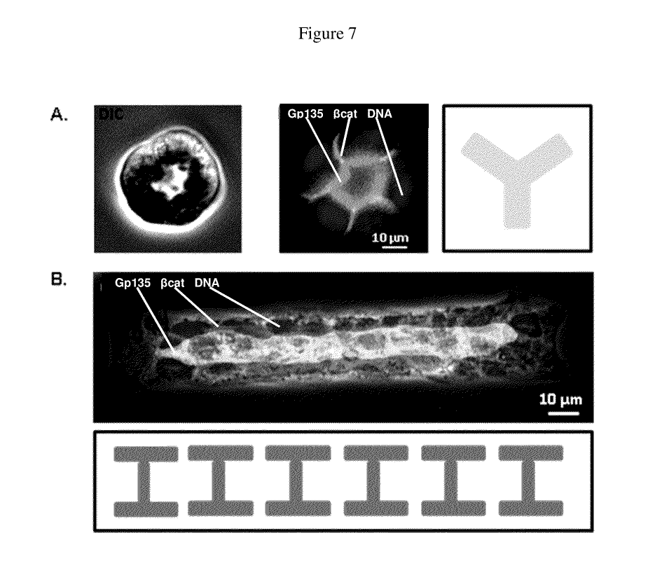

MDCK cells seeded onto laminin Y-shaped micropatterned adhesive supports formed acini with lumens after 2-3 days (FIG. 7A). Aggregates exhibited normalized polarity, with the apical surfaces of cells facing the lumen and the baso-lateral surfaces of cells facing other cells (FIG. 6).

Tubular structures were formed by seeding MDCK cells onto tubular micropatterned adhesive supports formed from multiple collagen-I H-shaped micropatterns of 200 um in length and having a length with ratio of 5:1 (FIG. 7B). The elongated tubes formed lumens after about 4 days (FIG. 7B) and also exhibited normalized polarity.

### Example 5

### Formation of Three-Dimensional Structures with Caco-2 Cells

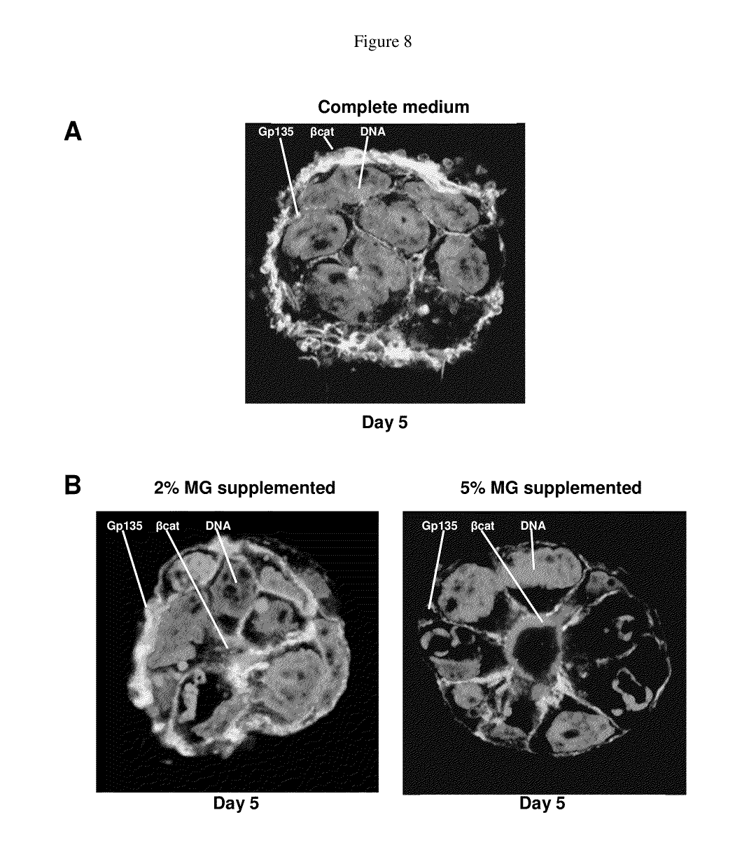

Caco-2 cells (C2BBel (clone of Caco-2 HTB-37) ATCC CRL-2102™ obtained from ATCC were prepared and seeded onto collagen-I disc-shaped micropatterned adhesive supports essentially as described in Examples 1-3 and allowed to form three-dimensional aggregates. Caco-2 cells were overlaid with either a solution of 2% or 5% Matrigel™ and complete culture medium comprising Dulbecco's Modified Eagle Medium (DMEM; Invitrogen) supplemented with 10% fetal bovine serum, 2 mM L-Glutamine and 1% penicillin-streptomycin or the complete culture medium only. Multicellular assemblies and lumen formation was evident after 5 days of culture (FIG. 8).

Cell aggregates were fixed on the support using paraformaldehyde 4% for 20 minutes and permeabilized with Triton-X100 0.1%-PBS solution. Supports were then incubated with PBS-BSA 3%, followed by incubation with a solution of PBS-BSA 3% with diluted β-catenin antibodies (Santa Cruz Biotechnology, Inc. 2145 Delaware Avenue, Santa Cruz, Calif. U.S.A.) and secondary antibodies. Supports were also incubated with DAPI for nuclear staining (DNA). TRITC-Phalloidin was used for actin microfilament staining (F-actin) which localizes at the apical brush border of epithelial cells, and thus serves as an apical polarity marker. Slides were mounted using ProLong-Gold® (Invitrogen) and then imaged using a Zeiss LSM710 confocal microscope. Images were acquired using ZEN 2010 software and then treated using ImageJ software.

Caco-2 cells overlaid with complete culture medium only (ie. medium without the addition of Matrigel™) formed follicles with inverted polarity (FIG. 8A). The addition of Matrigel™-supplemented medium induced Caco-2 acini formation after 3-5 days (FIG. 8B).

### Example 6

### Averaging of Internal Cell Organization

An array of six substantially identical 2-cell three-dimensional structures were created according to the methods described in Examples 1-3 on collagen-I H-shaped micropatterns.

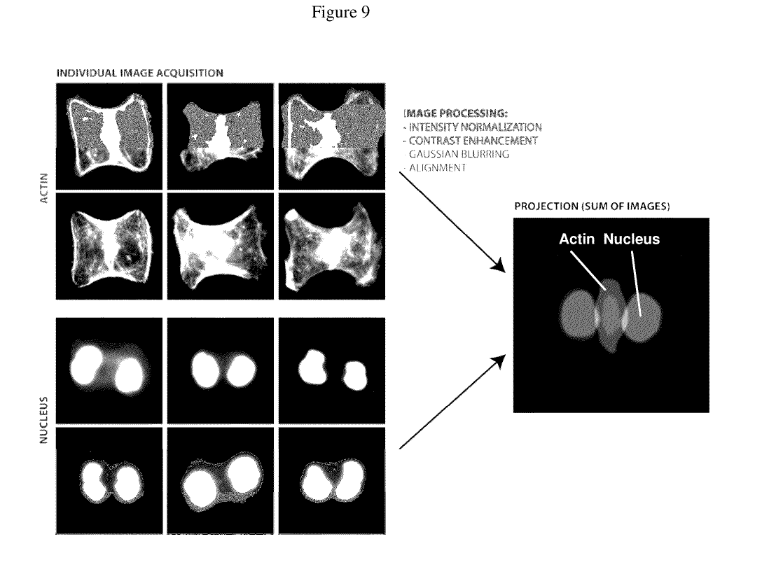

The resulting three-dimensional structures were treated with the fixative reagent 4% PFA and immunostaining was performed for different cell compartments. The apical membrane was stained with a primary antibody anti-GP135 and a secondary antibody anti-mouse IgG-DyLight 488 (Thermo Fisher Scientific Inc.). Actin was stained with phalloidin-FITC. The baso-lateral membrane was stained with a primary antibody anti-β-catenin and a secondary antibody anti-rabbit IgG-Cy3. Nucleus was stained with Hoechst. Images were acquired using confocal or wide-field deconvolution microscopy microscopy and transferred to an image analysis software (Image J). An array of at least 10 individual images of representative cells were created from the same sample. For each stained organelle a stack file of images was created. Translational alignment of stacked images was performed using the centroid of one of the nuclei as a spatial reference. Then, a projection of each stack of images was performed by summing individually the signal of each pixel in all of the stacks. The image resulting from such projection is then combined with the resulting image of each organelle using different color codes. Intensity measures, organelle distribution and other derived parameters are used for analysis (FIG. 9).

### Example 7

### Effect of Adhesive Proteins and Micropattern Size

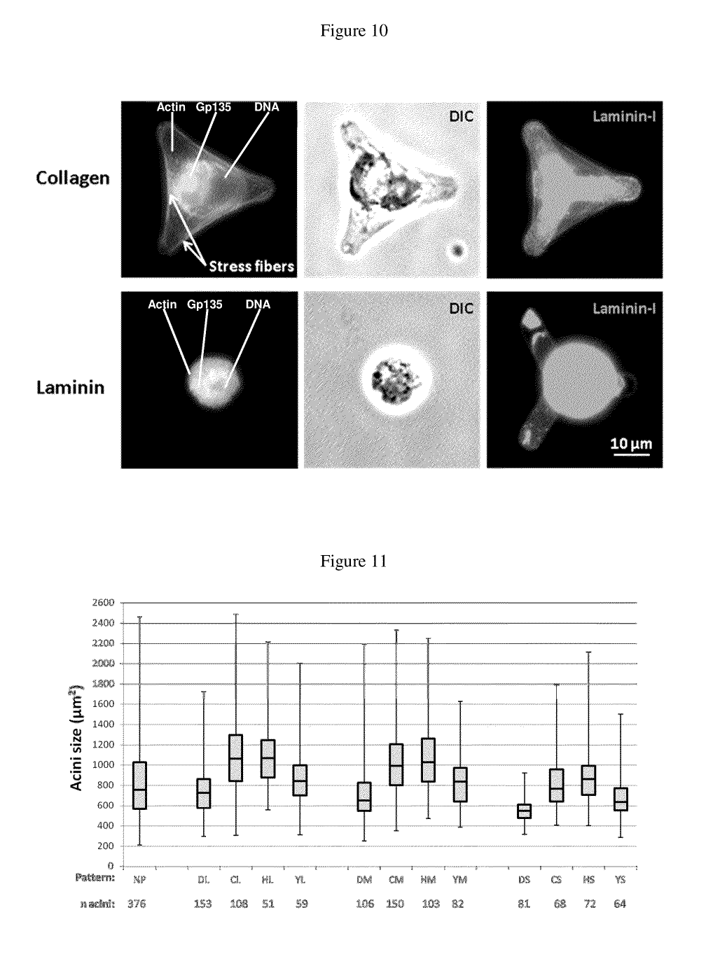

MDCK cells were prepared and seeded onto collagen-I or laminin Y-shaped micropatterns according to the methods described in Examples 1-3. The cells were fixed and stained 5 hours after seeding as described above. It was observed that the cell on the collagen-I micropattern spread, forming stress fibers to adopt the shape of the pattern, while the cell on the laminin micropattern was round, without stress fibers (FIG. 10).

Accordingly, depending on the adhesion proteins used to coat the micropatterns, cells behave differently, although the aggregates exhibit normalized polarity in all cases. This is thought to be due to the competition between cell spreading on the adhesive support and cell-to-cell adhesion. For the same reason, we have also observed an inverse correlation between micropattern size and speed of acini formation: on small micropatterns, cells tend to remain round and form acini more rapidly than the cells spread on large micropatterns.

Thus, by varying the protein for coating and/or the size of the different patterns/shapes a wide diversity of combinations is provided, which allows the selection of the most suitable multicellular arrangement for a given question or screen.

### Example 8

### Micropattern Normalization of 3D Cyst Size

MDCKII cells obtained from ECACC (Catalogue No: 00062107) were prepared for cell deposition on a laminin coated Starter's CYTOOchip as described in Example 2. Cells (20,000 per chip) were seeded without any further washing step. After 3 to 4 hours incubation in a cell incubator (37° C., 5% CO2) an overlay of 2.5% Matrigel™ was added as described in Example 3. At 72 hours of culture, 3-dimensional structures were fixed and stained as described in Example 4. Images were acquired on a CellInsight™ (Thermo Fischer Scientific) using a 10× objective and analyzed using the Morphology Explorer v4.0 BioApplication software. Spherical cysts with apico-basolateral polarity were positively identified based on the presence of a lumen (stained by apical membrane marker gp135) inside a region of interest defined by F-actin staining. Acini area (jm²) was calculated as the cross-sectional area from the 2D microscope image.

Quantification of the size distribution of the acini formed on each of the 4 different patterns and 3 different sizes available on a Starter's CYTOOchip showed that the Disc Small (DS 700 μm²) micropattern significantly reduced heterogeneity of cyst size within a population (FIG. 11). On the DS micropattern, a significant fraction (50%) of acini were observed in the size range 480-610 μm². The range of lumen size and number of cells per acini is also expected to be normalized under these conditions. In contrast, on non-patterned (NP) fully homogenous coating, 50% of acini have a broad size range varying from 560-1020 μm².

These results show that a normalized population of acini can be obtained by choosing certain micropattern shapes and sizes, without detrimentally affecting efficiency of lumen formation or polarity. We reason that compared to crossbow, H, and Y, the symmetrical fully adhesive disc provides a geometry and size that promotes uniformity during cyst formation. Variations on the disc shape and size may further improve normalization of the cyst population. The ability to obtain a population of homogenous sized acini in a reproducible manner is of great importance for complex 3D assays, directly influencing drug penetrance and thus interpretation of data and the robustness of pharmacological studies.

### REFERENCES

Herlyn et al. U.S. Pat. No. 7,217,570 B2
Kataoka et al. U.S. Patent 2004/0197907
Kataoka et al. U.S. Patent 2010/7691369B2
Sokabe et al. U.S. Patent 2010/0331216A1
Fang et al. U.S. Patent 2009/0298166A1
F. Xu et al., “A three-dimensional in vitro ovarian cancer coculture model using a high-throughput cell patterning platform”, Biotechnol. J. 2011, 6, 204-212
C. M. Williams et al., “Autocrine-Controlled Formation and Function of Tissue-Like Aggregates by Primary Hepatocytes in Micropatterned Hydrogel Arrays”, Tissue Engineering: Part A, Vol. 17, Nos 7 and 8, 2011
Coligan et al., 1999 “Current protocols in Protein Science” Volume I and II (John Wiley & Sons Inc.)

Ross et al., 1995 “Histology: Text and Atlas”, 3rd Ed., (Williams & Wilkins)
Kruse & Patterson (eds.) 1977 “Tissue Culture” (Academic Press)
Alberts et al. 2000 “Molecular Biology of the Cell” (Garland Science)
Bryant and Mostov, Nat Rev Mol Cell Biol. 2008 November; 9(11): 887-901
Bornens et al. WO 2005/026313
Théry et al., Cell Motil Cytoskeleton 2006, 63(6):341-55

1. A device comprising:
a support defining a surface;
at least one two-dimensional adhesive pattern on said surface, and
at least one three-dimensional multicellular assembly immobilised on said two-dimensional adhesive pattern, wherein said three-dimensional multicellular assembly has an organised structure with a normalised polarity.

2. A device comprising:
a support defining a surface;
at least two two-dimensional adhesive patterns on said surface, and
at least two three-dimensional multicellular assemblies immobilised on said two-dimensional adhesive patterns, wherein said three-dimensional multicellular assemblies have an organised structure with a normalised polarity, and wherein said three-dimensional multicellular assemblies have substantially the same structure, shape and size.

3. A device according to claim 1, wherein said three-dimensional multicellular assemblies comprise at least one lumen.

4. A device according to claim 3, wherein said three-dimensional assembly has a structure selected from the group consisting of an acinus and a tube.

5. A device according to claim 1, wherein said adhesive pattern is adapted to control initial cell spreading of the three-dimensional multicellular assembly.

6. A device according to claim 5, wherein said control of initial cell spreading is by at least one selected from the group consisting of topographically and chemically.

7. A device according to claim 1, wherein said adhesive pattern is approximately at least one shape selected from the group consisting of a disc, a crossbow, an H, a Y, a rectangle, a ring and an S.

8. A device according to claim 1, wherein said adhesive pattern is adapted to promote the formation of a tube.

9. A device according to claim 8, wherein said adhesive pattern has a length to width ratio of 10:1.

10. A device according to claim 8, wherein said approximately rectangular shape has a length of from about 100 to 300 μm, optionally from 200 to 300 μm.

11. A method of forming a three-dimensional multicellular assembly comprising:
(i) providing a support defining a surface, said support comprising at least one two-dimensional adhesive pattern on said surface, wherein said adhesive pattern is adapted to control initial cell spreading of the three-dimensional multicellular assembly;
(ii) seeding said support with cells; and
(iii) culturing said cells under conditions and for a sufficient period of time to obtain a three-dimensional multicellular assembly on said two-dimensional adhesive pattern, wherein said three-dimensional multicellular assembly has an organised structure with a normalised polarity.

12. A method according to claim 11, wherein (iii) comprises overlaying said cells with a culture medium.

13. A method according to claim 12, wherein said culture medium comprises extracellular matrix proteins.

14. A method of defining an average cell comprising:
(i) providing a support defining a surface, said support comprising at least one two-dimensional adhesive pattern on said surface, wherein said adhesive pattern is adapted to control initial cell spreading of the three-dimensional multicellular assembly;
(ii) seeding said support with cells; and
(iii) forming organised three-dimensional multicellular assemblies with a normalised polarity on said two-dimensional adhesive pattern using the method of claim 11, wherein said three-dimensional multicellular assemblies have substantially the same structure, shape and size; and
(iv) imaging said three-dimensional multicellular assemblies and defining an average cell by averaging data obtained from images of said three-dimensional multicellular assemblies.

15. A device for forming a three-dimensional multicellular tubular structure comprising:
a support defining a surface;
at least one two-dimensional adhesive pattern on said surface, wherein said adhesive pattern is adapted to form a tubular structure.
